(19) United States
(12) Patent Application Publication
(10) Pub. No.: US 2021/0015519 A1
(43) Pub. Date: Jan. 21, 2021

(54) SURGICAL PORT MANIPULATOR

(71) Applicant: Covidien LP, Mansfield, MA (US)

(72) Inventors: Dwight Meglan, Westwood, MA (US); Renen Bassik, Lexington, MA (US)

(21) Appl. No.: 17/041,563

(22) PCT Filed: Apr. 1, 2019
(86) PCT No.: PCT/US2019/025094

§ 371 Date: Sep. 25, 2020

Related U.S. Application Data
(60) Provisional application No. 62/660,425, filed on Apr. 20, 2018.

(51) Int. Cl. A61B 17/34; A61B 90/50; A61B 34/30

(52) U.S. Cl. CPC A61B 17/3423; A61B 2090/065; A61B 34/30; A61B 90/50

(57) ABSTRACT

A surgical port manipulator includes a body housing a motion source, an arm coupled to the body, a load sensor associated with the arm, and a controller in communication with the load sensor and the motion source. The arm has an end configured to rotatably couple a surgical port thereto such that the surgical port is rotatable relative to the arm in at least two degrees of freedom in response to a supply of power from the motion source. The load sensor is configured to sense a load exerted on the surgical port. The controller is configured to direct the motion source to move the surgical port in a direction in response to the load sensor sensing a threshold load oriented in the direction.

### BACKGROUND

Robotic surgical systems have been used in minimally invasive medical procedures. Some robotic surgical systems include a robot arm having an instrument drive assembly coupled thereto for coupling surgical instruments to the robot arm, such as, for example, a pair of jaw members, electrosurgical forceps, cutting instruments, or any other endoscopic or open surgical devices. In some robotic surgical systems, a trocar or a surgical port may be provided to assist in accessing a surgical site.

Prior to or during use of the robotic system, surgical instruments are selected and connected to the instrument drive assembly of each robot arm, where the instrument drive assembly can drive the actuation of an end effector of the surgical instrument. Under certain procedures, a surgical port may be positioned within a small incision in a patient. During a procedure, the end effector and/or a portion of the surgical instrument may be inserted through the surgical port, and the small incision in the patient, to bring the end effector proximate a working site within the body of the patient. Such surgical ports provide pressure sealing during insufflation of the body cavity of the patient and may act as a guide channel for the surgical instrument during insertion and actuation of the end effector.

During a surgical procedure, the surgical instrument may contact an inner sidewall of the surgical port, which may prevent or resist movement of the surgical instrument to a particular location within the surgical site due to resistance exerted by the tissue surrounding the surgical port. Accordingly, there is a need to reduce the amount of resistance the surgical port, and the surrounding tissue, exerts on the surgical instrument during movement of the surgical instrument within the surgical port.

### SUMMARY

According to an aspect of the present disclosure, a surgical port manipulator includes a body housing a motion source, an arm coupled to the body, a load sensor associated with the arm, and a controller in communication with the motion source and the load sensor. The arm has an end portion configured to rotatably couple a surgical port thereto such that the surgical port is rotatable relative to the end portion of the arm in at least two degrees of freedom (DOF). The arm is configured to move the surgical port in response to a supply of power from the motion source. The load sensor is configured to sense a load exerted on the surgical port. The controller is configured to direct the motion source to move the surgical port in a first direction or a second direction in response to the load sensor sensing a threshold load oriented in the first direction or the second direction.

In some embodiments, the controller may be configured to continue directing the motion source to move the surgical port until the load sensor ceases sensing the threshold load.

It is contemplated that the controller may be configured to direct the motion source to move the surgical port in the first direction upon the load sensor sensing a load oriented in the first direction. The controller may be configured to direct the motion source to move the surgical port in the second direction upon the load sensor sensing a load oriented in the second direction.

It is envisioned that the first direction may be in a first DOF, and the second direction may be in a second DOF. The first DOF may be a pitch rotation such that the surgical port rotates about a first transverse axis defined therethrough that is perpendicular to a longitudinal axis defined by the arm in response to the load sensor sensing the threshold load in the first direction. The second DOF may be a roll rotation such that in response to the load sensor sensing the threshold load in the second direction, the surgical port rotates about a second transverse axis, which is perpendicular to the first transverse axis and parallel with the longitudinal axis of the arm.

In some embodiments, the end portion of the arm may include a multi-DOF remote center of motion (“RMC”) assembly. The end portion of the arm may include a coupler connected to the RCM assembly and configured to releasably attach to a surgical port. The coupler may be movable relative to another end portion of the arm in the at least two DOFs via the RCM assembly. The coupler may have an arcuate shape and may be dimensioned to engage an outer surface of the surgical port.

It is contemplated that the arm may include a plurality of linkages rotatably coupled to one another.

It is envisioned that the body may be configured to be mounted to a surgical bed.

In another aspect of the present disclosure, a robotic surgical system is provided and includes a surgical robotic arm for supporting and moving a surgical instrument, a surgical port for providing access to a surgical site, and the surgical port manipulator.

### BRIEF DESCRIPTION OF THE DRAWINGS

Embodiments of the present disclosure are described herein with reference to the accompanying drawings, wherein:

FIG. 1 is a schematic illustration of a robotic surgical system in accordance with the principles of the present disclosure;

FIG. 2 is a schematic side view of the robotic surgical system of FIG. 1, illustrating a surgical port manipulator thereof and a cart supporting a robot arm of the robotic surgical system;

FIG. 3 is a top view of the surgical port manipulator of FIG. 2; and

FIG. 4 is a side view of the surgical port manipulator of FIG. 2.

### DETAILED DESCRIPTION

Embodiments of the presently disclosed robotic surgical system including the surgical port manipulator thereof are described in detail with reference to the drawings, in which like reference numerals designate identical or corresponding elements in each of the several views. As is used in the art, the term “distal” refers to a portion of the robotic surgical system, which is farther from the user, and the term “proximal” refers to a portion of the robotic surgical system, which is closer to the user.

The present disclosure provides a surgical port manipulator for assisting a clinician or robot in manipulating a surgical instrument through a surgical port or access port fixed within an incision. During a surgical procedure, an attempt to adjust the spatial orientation of a surgical port fixed within an incision may be met with resistance by the surrounding tissue. For example, when a manipulation of a surgical instrument results in the surgical instrument meeting an inner sidewall of the surgical port, further manipulation of the surgical instrument may be difficult due to a reaction force exerted on the surgical port and, in turn, the surgical instrument, by the surrounding tissue. The active motion surgical port manipulator of the present disclosure assists in overcoming these reaction forces.

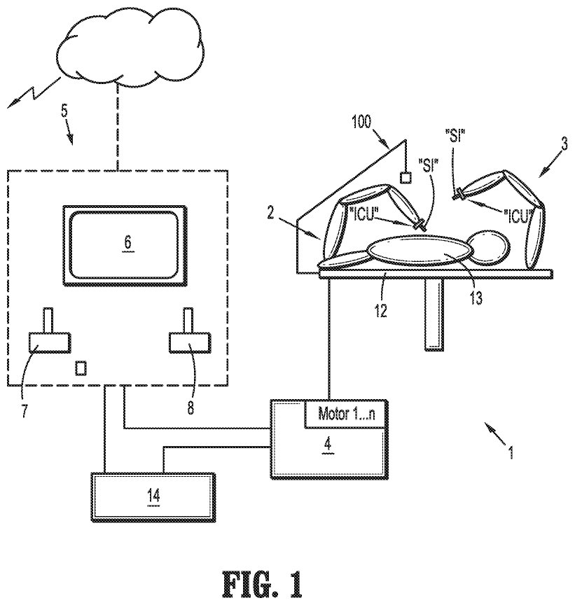

Referring initially to FIG. 1, a medical work station or robotic surgical system is shown generally as robotic surgical system 1 and generally includes a plurality of robot arms 2, 3; a control device 4; an operating console 5 coupled with control device 4; and a surgical port manipulator 100. Operating console 5 includes a display device 6, which is set up in particular to display three-dimensional images; and manual input devices 7, 8, by means of which a person (not shown), for example a surgeon, is able to telemanipulate robot arms 2, 3 in a first operating mode, as known in principle to a person skilled in the art.

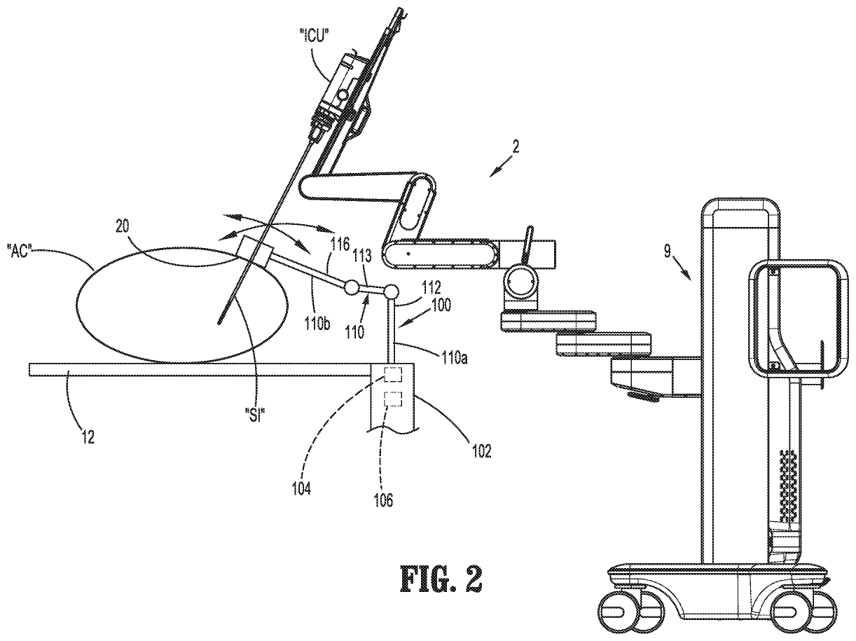

Each of the robot arms 2, 3 may be supported by a respective cart 9 (FIG. 2), and may include a plurality of members, which are connected through joints, and an instrument control unit “ICU”, to which may be attached, for example, an instrument drive assembly of a surgical instrument “SI.” The surgical instrument “SI” supports an end effector (not shown) including, for example, a pair of jaw members, electrosurgical forceps, cutting instruments, or any other endoscopic or open surgical devices. For a detailed discussion and illustrative examples of the construction and operation of an end effector for use with instrument control unit “ICU”, reference may be made to commonly owned International Patent Application No. WO/2015/088647, filed on Oct. 20, 2014, and entitled “Wrist and Jaw Assemblies for Robotic Surgical Systems,” and U.S. Provisional Patent Application No. 62/341,714, filed on May 26, 2016, entitled “Robotic Surgical Assemblies,” the entire content of each of which being incorporated herein by reference.

Robot arms 2, 3 may be driven by electric drives (not shown) that are connected to control device 4. Control device 4 (e.g., a computer) is set up to activate the drives, in particular by means of a computer program, in such a way that robot arms 2, 3, instrument control units “ICU”, and thus the surgical instruments “SI” execute a desired movement or articulation according to a movement defined by means of manual input devices 7, 8. Control device 4 may also be set up in such a way that it regulates the movement of robot arms 2, 3 and/or of the drives.

Robotic surgical system 1 is configured for use on a patient 13 lying on a patient table 12 to be treated in an open surgery, or a minimally invasive manner, by means of surgical instrument “SI.” Robotic surgical system 1 may also include more than two robot arms 2, 3, the additional robot arms likewise being connected to control device 4 and being telemanipulatable by means of operating console 5. An instrument control unit and a surgical instrument may also be attached to the additional robot arm. Robotic surgical system 1 may include a database 14 coupled to or with control device 4, in which pre-operative data from patient 13 and/or anatomical atlases, for example, may be stored.

Control device 4 may control a plurality of motors (Motor 1 . . . n). Motors (Motor 1 . . . n) may be part of instrument control unit “ICU” and/or disposed externally of instrument control unit “ICU”. In use, as motors (Motor 1 . . . n) are driven, movement and/or articulation of the instrument drive assembly of surgical instrument “SI”, and an end effector attached thereto, is controlled. It is further envisioned that at least one motor (Motor 1 . . . n) receives signals wirelessly (e.g., from control device 4). It is contemplated that control device 4 coordinates the activation of the various motors (Motor 1 . . . n) to coordinate an operation, movement, and/or articulation of robot arms 2, 3 and/or surgical instrument “SI.” It is envisioned that each motor may correspond to a separate degree of freedom of robot arms 2, 3, and/or surgical instrument “SI” engaged with instrument control unit “ICU.” It is further envisioned that more than one motor, including every motor (Motor 1 . . . n), is used for each degree of freedom.

For a detailed discussion of the construction and operation of an exemplary medical work station, reference may be made to U.S. Pat. No. 8,828,023, filed on Nov. 3, 2011, and entitled “Medical Workstation,” the entire content of which is incorporated herein by reference.

With reference to FIGS. 2-4, the active motion surgical port manipulator 100 of the robotic surgical system 1 supports and drives a movement of a surgical port 20 or trocar that provides access into a surgical site within a patient, such as, for example, an abdominal cavity “AC” or a thoracic cavity. The surgical port manipulator 100 includes a hub or main body 102 and an arm 110 coupled to the main body 102. The main body 102 houses a motion source 104, such as, for example, a power source, and a controller 106 and may be configured to be detachably coupled to a surface in an operating room such as a side of a surgical bed 12. For example, the main body 102 may have a clip, adhesive, a hook, or any suitable mechanism for detachably coupling the manipulator 100 to a surgical bed 12, a cart (e.g., robotic arm cart 9), a wall, a ceiling, or the like. The motion source 104 housed within the main body 102 may be an electric motor, a pneumatic power source, a hydraulic power source, or the like.

The arm 110 of the surgical port manipulator 100 has a first end portion 110a coupled to the main body 102, and a second end portion 110b configured to rotatably couple a surgical port 20 thereto. In embodiments, the surgical port manipulator 100 may be devoid of the main body 102, and the first end portion 110a of the arm 110 may instead include the motion source 104 and the controller 106 such that the first end portion 110a of the arm 110 may be directly coupled to a surgical bed 12 or other surface in an operating room.

Similar to the robot arm 2 (FIG. 1), the arm 110 of the surgical port manipulator 100 may include a plurality of linkages 112, 113, 116, which are connected through joints. Each of the linkages 112, 113, 116 may be driven by electric drives (not shown) that are connected to the controller 106 of the main body 102. The controller 106 may be set up to activate the drives, in particular by means of a computer program, in such a way that the arm 110 of the surgical port manipulator 100 executes a desired movement.

With continued reference to FIGS. 2-4, the second end portion 110b of the arm 110 is configured to rotatably support a surgical port 20 such that the surgical port 20 is rotatable/pivotable relative to the second end portion 110b of the arm 110 in a plurality of degrees of freedom (DOF) (e.g., pitch, yaw, roll) in response to a supply of power from the motion source 104. The second end portion 110b of the arm 110 includes a multi-DOF remote center of motion (“RCM”) assembly 114 and a coupler or connector 120 operably coupled to the RCM assembly 114. The RCM assembly 114 may incorporate any of the RCM mechanisms described in “Kinematic Design Considerations for Minimally Invasive Surgical Robots: An Overview,” Kuo et al., The International Journal of Medical Robotics and Computer Assisted Surgery (2012), and “Remote Center of Motion (RCM) Mechanisms for Surgical Operations,” Aksungur et al., International Journal of Applied Mathematics, Electronics and Computers (2014), the entire contents of each of which being incorporated by reference herein.

The RCM assembly 114 is movable relative to a distal linkage 116 of the arm 110 in a plurality of DOFs such as a pitch rotation, a yaw rotation, and a roll rotation. In some embodiments, the RCM assembly 114 may be disposed adjacent the first end portion 110a of the arm 110 rather than between the surgical port 20 and the second end portion 110b of the arm 110. The RCM assembly 114 is operably coupled to the motion source 104 for driving the movement of the RCM assembly 114. In one embodiment, the RCM assembly 114 may rotate in the plurality of DOFs by utilizing a plurality of pulleys 115 and cables 117 similar to a wrist assembly described in co-owned International Patent Application No. WO/2015/088647, filed on Oct. 20, 2014, the entire content of which already incorporated by reference above.

The coupler 120 of the second end portion 110b of the arm 110 is movable (e.g., rotatable/pivotable) relative to the distal linkage 116 of the arm 110 in a plurality of DOFs (e.g., pitch, yaw, roll) via the RCM assembly 114. The coupler 120 includes a bracket 122 and a pair of flexible clamp arms 124a, 124b extending from the bracket 122. The flexible clamp arms 124a, 124b each have an arcuate shape to accommodate a circular surgical port (e.g., surgical port 20). In embodiments, the clamp arms 124a, 124b may have any suitable shape (e.g., linear) to accommodate variously shaped surgical ports. The clamp arms 124a, 124b of the coupler 120 are flexible to fit over an outer surface of the surgical port 20 to snap-fittingly engage and retain the surgical port 20 therebetween. In embodiments, the coupler 120 may detachably couple to the surgical port 20 via any suitable engagement mechanism, such as, for example, friction-fit.

The coupler 120 may include a high-friction material (e.g., rubber) lining an inner surface 126 of the clamp arms 124a, 124b to enhance the strength of the connection between the surgical port 20 and the coupler 120. In embodiments, the coupler 120 may be configured as a clamp, a fastener (e.g., a screw threaded into the surgical port 20), or any suitable coupling mechanism that releasably or fixedly couples a surgical port to the surgical port manipulator 100. In embodiments, the surgical port manipulator 100 may be devoid of the coupler 120 such that the surgical port 20 may be directly connected to the RCM assembly 14 rather than being indirectly connected to the RCM assembly 14 via the coupler 120.

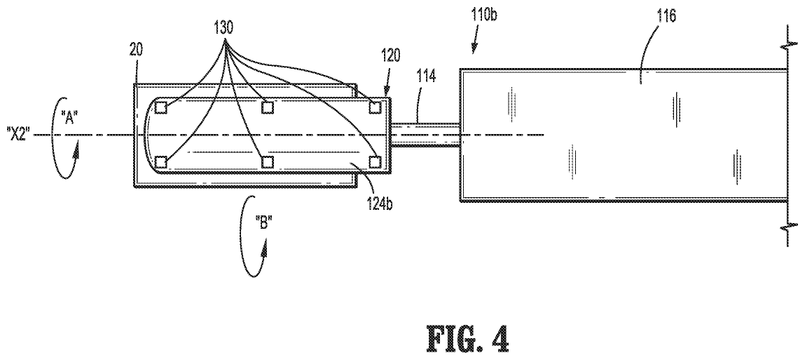

With continued reference to FIGS. 2-4, the surgical port manipulator 100 includes a plurality of load sensors 130 associated with the second end portion 110b of the arm 110 for sensing a load or loads exerted on the attached surgical port 20. For example, the load sensors 130 may be disposed on the inner surface 126 of the coupler 120 in an annular array such that a load exerted on the coupler 120 in any direction will be sensed by a multiplicity of the load sensors 130. In embodiments, the load sensors 130 may be situated in any suitable array on or in the coupler 120 and/or may be disposed in stacked rows on the coupler 120 (see FIG. 4). In some embodiments, the load sensors 130 may be strain-sensing resistors, strain and/or pressure sensing MEMS devices, torque sensors, strain gauges, light sensors, photodetectors, or the like. In other embodiments, the load sensors 130 may be associated with various portions of the surgical port manipulator 100, such as, for example, the RCM assembly 114, the linkages 112, 114, 116 of the arm 110, and/or the surgical port 20.

The controller 106 housed in the main body 102 of the surgical port manipulator 100 includes a processor (not shown) operably connected to a memory, which may include transitory type memory (e.g., RAM) and/or non-transitory type memory (e.g., flash media, disk media, etc.). The processor of the controller 106 includes an output port that is operably connected to the motion source 104 allowing the processor to control the output of the motion source 104 according to either open and/or closed control loop schemes. A closed loop control scheme is a feedback control loop, in which the load sensors 130 measure a load and provide feedback to the controller 106. The controller 106 is configured to then signal the motion source 104, which adjusts the power supplied to the RCM assembly 114. Those skilled in the art will appreciate that the processor may be substituted by using any logic processor (e.g., control circuit) adapted to perform the calculations and/or set of instructions described herein including, but not limited to, field programmable gate arrays, digital signal processor, and combinations thereof.

The controller 106 of the surgical port manipulator 100 is configured to adjust the amount of power supplied by the motion source 104 to the RCM assembly 114 based on the loads sensed by the load sensors 130. In particular, the controller 106 is configured to direct the motion source 104 to effect a rotation of the coupler 120 via the RCM assembly 114 in a specific direction in response to the load sensors 130 sensing a threshold load oriented in the specific direction. In this way, the coupler 120 will move the attached surgical port 20 in the direction that the load applied on the surgical port 20 is oriented, as will be described in detail below. The controller 104 is further configured to adjust the spatial orientation of the attached surgical port 20 while maintaining the remote center of motion of the surgical port 20. As such, the load sensors 130 may sense transverse/shear forces and/or bending moments applied on the surgical port 20 via the surgical instrument “SI,” and in response the controller 106 effects a movement of the coupler 120 and, in turn, the surgical port 20, in a rotational direction about the remote center of motion.

In operation, a surgical port (e.g., surgical port 20) is positioned within an incision formed in tissue (e.g., an abdominal cavity “AC”) of a patient to provide access for surgical instruments into a surgical site within the patient's body. The arms 124a, 124b of the coupler 120 of the surgical manipulator 100 are positioned around the surgical port 20. In embodiments, the surgical port 20 may be attached to the coupler 120 of the surgical port manipulator 100 prior to the surgical port 20 being positioned into incision. A surgical instrument “SI” (e.g., a surgical stapler), which may be attached to the robotic arm 2 (FIG. 1), is passed through the surgical port 20 and into the surgical site. In embodiments, the surgical instrument “SI” may be manually positioned and manipulated within the surgical port 20 rather than be attached to the robotic arm 2.

During the natural course of a surgical procedure, the surgical instrument “SI” may come into contact with an inner sidewall of the surgical port 20. Due to the surgical port 20 being surrounded by tissue, further movement of the surgical instrument “SI” toward a target location within the surgical site is met with resistance by the surrounding tissue. If this occurs, the robotic arm 2 or a clinician may require assistance from the surgical port manipulator 100 to move the surgical port 20 out of the way of the surgical instrument “SI” and against the resistance of the surrounding tissue.

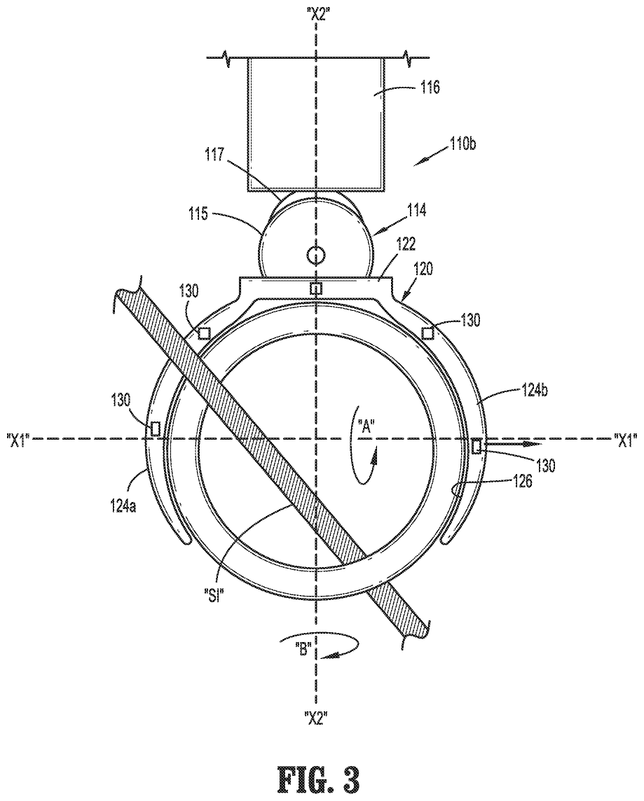

For example, if the surgical instrument “SI” makes contact with a portion of the surgical port 20 resulting in a rotational moment on the surgical port 20 oriented in a rotational direction “A,” shown in FIGS. 3 and 4, this is an indication that a pitch angle of the surgical port 20 needs to be adjusted to allow for the continued movement of the surgical instrument “SI” toward its target location. Accordingly, if and when the rotational moment oriented in the direction “A,” sensed by the load sensor(s) 130, exceeds a threshold load (e.g., a load greater than a nominal load indicative of something more than incidental contact of the surgical instrument “SI” with surgical port 20), the load sensor(s) 130 senses this load and sends a signal to the controller 106. The controller 106 in turn directs the motion source 104 to activate the RCM assembly 114 to rotate the coupler 120 and the attached surgical port 20 in the direction “A,” about a first horizontal axis “X1” (FIG. 3) defined transversely through the surgical port 20 that is perpendicular to a longitudinal axis “X2” defined by the distal linkage 116 of the arm 110.

Rotating the surgical port 20 in the direction “A” changes the pitch angle of the surgical port 20 to clear the way for the continued movement of the surgical instrument “SI” by moving the portion of the surgical port 20 that is blocking the desired movement of the surgical instrument “SI” in the same direction that the surgical instrument “SI” is being moved. The controller 106 continues to adjust the pitch angle of the surgical port 20 within the incision until the load sensors 130 cease sensing the threshold load being applied in the direction “A.”

If the surgical instrument “SI” makes contact with the surgical port 20 resulting in a load on the surgical port 20 oriented in a direction “B,” shown in FIGS. 3 and 4, this is an indication that a yaw or roll angle of the surgical port 20 needs to be adjusted to allow for the continued movement of the surgical instrument “SI” toward its target location. Accordingly, if and when the load oriented in the direction “B” exceeds the threshold load, the load sensor(s) 130 senses this load and sends a signal to the controller 106. The controller 106 in turn directs the motion source 104 to activate the RCM assembly 114 to rotate the coupler 120 and the attached surgical port 20 in the direction “B,” shown in FIG. 4, about the longitudinal axis “X2” defined by the distal linkage 116 of the arm 110.

Rotating the surgical port 20 in the direction “B” changes the yaw or roll angle of the surgical port 20 to clear the way for the continued movement of the surgical instrument “SI” by moving the portion of the surgical port 20 that is blocking the desired movement of the surgical instrument “SI” in the same direction that the surgical instrument “SI” is being moved. The controller 106 continues to adjust the yaw or roll angle of the surgical port 20 within the incision until the load sensors 130 cease sensing the threshold load being applied in the direction “C.”

In embodiments, the spatial orientation of the surgical port 20 within the incision may be adjusted by a clinician using telemanipulation rather than automatically by the controller 104.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

1. A surgical port manipulator, comprising:
a body housing a motion source;
an arm having a first end portion coupled to the body and a second end portion configured to rotatably couple a surgical port thereto such that the surgical port is rotatable relative to the second end portion of the arm in at least two degrees of freedom (DOF), the arm configured to move the surgical port in response to a supply of power from the motion source;
a load sensor associated with the arm and configured to sense a load exerted on the surgical port; and
a controller in communication with the motion source and the load sensor, wherein in response to the load sensor sensing a threshold load oriented in a first direction or a second direction, the controller is configured to direct the motion source to move the surgical port in at least one of the first direction or the second direction.

2. The surgical port manipulator according to claim 1, wherein the controller is configured to continue directing the motion source to move the surgical port until the load sensor ceases sensing the threshold load.

3. The surgical port manipulator according to claim 1, wherein the controller is configured to direct the motion source to move the surgical port in the first direction upon the load sensor sensing a load oriented in the first direction, and wherein the controller is configured to direct the motion source to move the surgical port in the second direction upon the load sensor sensing a load oriented in the second direction.

4. The surgical port manipulator according to claim 1, wherein the first direction is in a first DOF of the at least two DOFs, and the second direction is in a second DOF of the at least two DOFs.

5. The surgical port manipulator according to claim 4, wherein the first DOF is a pitch rotation such that the surgical port rotates about a first horizontal axis defined transversely therethrough that is perpendicular to a longitudinal axis defined by the arm in response to the load sensor sensing the threshold load in the first direction, and wherein the second DOF is a roll rotation such that the surgical port rotates about a second horizontal axis defined transversely therethrough that is parallel with the longitudinal axis of the arm in response to the load sensor sensing the threshold load in the second direction.

6. The surgical port manipulator according to claim 1, wherein the second end portion of the arm includes a remote center of motion (RCM) assembly.

7. The surgical port manipulator according to claim 6, wherein the second end portion of the arm further includes a coupler connected to the RCM assembly and configured to releasably attach to a surgical port.

8. The surgical port manipulator according to claim 7, wherein the coupler is movable relative to the first end portion of the arm in the at least two DOFs via the RCM assembly.

9. The surgical port manipulator according to claim 7, wherein the coupler has an arcuate shape and is dimensioned to engage an outer surface of the surgical port.

10. The surgical port manipulator according to claim 1, wherein the arm includes a plurality of linkages rotatably coupled to one another.

11. The surgical port manipulator according to claim 1, wherein the body is configured to be mounted to a surgical bed.

12. A robotic surgical system, comprising:
a surgical robotic arm for supporting and moving a surgical instrument;
a surgical port for providing access to a surgical site; and
a surgical port manipulator including:
a body housing a motion source;
an arm having a first end portion coupled to the body and a second end portion, the surgical port rotatably coupled to the second end portion of the arm such that the surgical port is rotatable relative to the second end portion of the arm in at least two degrees of freedom (DOF), the arm configured to move the surgical port in response to a supply of power from the motion source;
a load sensor configured to sense a load exerted on the surgical port; and
a controller in communication with the motion source and the load sensor, wherein in response to the load sensor sensing a threshold load oriented in a first direction or a second direction, the controller is configured to direct the motion source to move the surgical port in at least one of the first direction or the second direction.

13. The robotic surgical system according to claim 12, wherein the controller is configured to continue directing the motion source to move the surgical port until the load sensor ceases sensing the threshold load.

14. The robotic surgical system according to claim 12, wherein the controller is configured to direct the motion source to move the surgical port in the first direction upon the load sensor sensing a load oriented in the first direction, and wherein the controller is configured to direct the motion source to move the surgical port in the second direction upon the load sensor sensing a load oriented in the second direction.

15. The robotic surgical system according to claim 12, wherein the first direction is in a first DOF of the at least two DOFs, and the second direction is in a second DOF of the at least two DOFs.

16. The surgical port manipulator according to claim 15, wherein the first DOF is a pitch rotation such that the surgical port rotates about a first horizontal axis defined transversely therethrough that is perpendicular to a longitudinal axis defined by the arm in response to the load sensor sensing the threshold load in the first direction, and wherein the second DOF is a roll rotation such that the surgical port rotates about a second horizontal axis defined transversely therethrough that is parallel to the longitudinal axis of the arm in response to the load sensor sensing the threshold load in the second direction.

17. The robotic surgical system according to claim 12, wherein the second end portion of the arm includes a multi-DOF RCM assembly.

18. The robotic surgical system according to claim 17, wherein the second end portion of the arm further includes a coupler connected to the RCM assembly and configured to releasably attach to a surgical port.

19. The robotic surgical system according to claim 17, wherein the coupler is movable relative to the first end portion of the arm in the at least two DOFs via the RCM assembly.

20. The robotic surgical system according to claim 16, wherein the coupler has an arcuate shape and is dimensioned to engage an outer surface of the surgical port.
